(19) United States
(12) Patent Application Publication
(10) Pub. No.: US 2005/0260557 A1
(43) Pub. Date: Nov. 24, 2005

(54) Method of quantifying the cell-binding properties of a medical device

(76) Inventor: Mary Zacour, Pointe Claire (CA)

Correspondence Address: TORYS LLP;TD Centre, 79 Wellington Street West, Suite 3000, Box 270, Toronto, ON, M5K 1N2, CA

(21) Appl. No.: 11/118,398

(22) Filed: May 2, 2005

Related U.S. Application Data
(60) Provisional application No. 60/572,766, filed on May 21, 2004.

(51) Int. Cl. C12Q 1/00

(52) U.S. Cl. 435/4

(57) ABSTRACT

The invention provides a method for quantifying the cell-binding properties of a medical device. In practicing the method, a medical device having at least one type of binding agent is incubated with cells expressing a ligand having an affinity for the binding agent on the medical device. The cells that bind to the medical device are labeled with at least one marker. The marker is detected, and the quantity of cells bound to the medical device is determined. Alternatively, labeled cells having at least one type of ligand expressing an affinity for the at least one type of binding agent may be provided that are incubated with the medical device. The relative expression of the ligand on the cell line having an affinity for the binding agent is also determined. The method can be used to assess, in vitro, how well cells will bind to the medical device, thereby providing useful insight as to the effectiveness of the medical device to promote cell adherence to the device, prior to in vivo implantation of such devices.

### FIELD OF THE INVENTION

The invention generally relates to binding agents on medical devices. More particularly, this invention is concerned with measuring the cell-binding properties of engineered surfaces of medical devices by detecting the quantity of cells bound to the medical devices.

### BACKGROUND OF THE INVENTION

Medical devices are prevalently used to repair or replace damaged vessels, tissues, organs, and other structures to significantly improve the long-term outcome of patients. For example, approximately one-third of patients with coronary artery disease, a progressive disease that is a leading cause of death, are treated with interventional medical devices, such as stents (Michaels, et al. Circulation. 2002;106:187). Stents as shown in FIG. 1, are small, metal, mesh tubes used to prop open blocked arteries caused by hardening or atherosclerosis.

Atherosclerosis occurs when a buildup of deposits, such as cholesterol and fatty substances, accumulates in the inner lining of an artery. The buildup, also known as plaque, hardens and narrows the artery so that blood flow through the artery is significantly reduced. Blood clots may form, and if blood flow is blocked to the heart, it can cause a heart attack while clots that prevent flow to the brain can cause a stroke.

Alternatives to bypass surgery are often used to improve blood flow. Blocked arteries can be opened with a procedure known as Percutaneous Transluminal Coronary Angioplasty (PTCA) or balloon angioplasty. In an angioplasty procedure, a catheter that has a small, inflatable balloon at the tip is inserted and guided to the site of obstruction. The balloon is expanded, opening the artery by pushing the plaque buildup into the artery wall. When injury to the lining of the artery occurs from balloon angioplasty, a complex series of inflammatory events and tissue remodeling can ensue, which may culminate in thrombosis and often leads to re-narrowing or restenosis of the artery.

Stents can be inserted into the blocked arteries to reduce the occurrence of acute and subacute restenosis. The stent is then deployed and acts like a scaffold to keep the vessel open and can be used either in place of or along with an angioplasty. Compared with balloon angioplasty, where the chance of restenosis is 40%, stents reduce the chance of restenosis to 25% (Dangas et. al. Circulation. 2002;105:2586). Stents can also be used to prevent strokes by opening blocked carotid arteries in the neck that supply blood to the brain, or they can be used to keep open other blocked passages, such as the esophagus, ureter, and bile duct.

Since restenois can also occur with implanted medical devices, the medical devices can be coated with therapeutic agents to help prevent reclosure of passageways and allow blood to flow over the device without clotting. These agents can be chemical, such as heparin or phosphorylcholine, that help prevent thrombosis and/or the inflammatory response that leads to restenosis, or they can be cytotoxic drugs, such as paclitaxel, that inhibit restenosis by preventing cell proliferation, or they can mimic tissue scaffold, such as collagen, to promote rapid healing, or they can be binding agents, such as antibodies or antigens, that recruit specific cells to the site of the injury caused by stent insertion and promote new tissue, for example endothelial cells, to cover the medical device.

U.S. patent application Ser. No. 2004/0029268, incorporated herein by reference, discusses a technique for re-endothelializing an artery whose endothelial layer has been damaged by balloon angioplasty. A multispecific antibody is introduced into the bloodstream of a patient, preferably prior to angioplasty. The multispecific antibody binds to a first antigen binding site directed against a surface marker on endothelial progenitor cells and also to a second antigen binding site directed against a subendothelial epitope. Once the angioplasty is performed and the target epitopes on the subendothelium have been exposed, the multispecific antibodies already bound to the endothelial progenitor cells also bind to the subendothelium. The cells then proliferate and cover the exposed subendothelium.

Coating medical devices with therapeutic agents is well known to one of ordinary skill in the art. In U.S. Pat. No. 6,231,600, incorporated herein by reference, a restenosis inhibiting coating of Taxol and a clot preventing coating of Heparin are applied onto a stent. Methods of coating an implantable device are described in U.S. Patent Application Publication 2003/0157241, U.S. Pat. No. 6,641,611,and U.S. Pat. No. 6,569,195, which are incorporated herein by reference. In U.S. patent application Ser. No. 2002/0049495, incorporated herein by reference, a medical device is coated with an antibody that reacts with a surface ligand on circulating progenitor cells to promote attachment and subsequent proliferation of progenitor endothelial cells on a medical device. U.S. Pat. No. 6,656,966, incorporated herein by reference, discusses the use of nitrosated or nitrosylated taxanes as therapeutic agents to prevent and/or treat restenosis and atherosclerosis. In U.S. patent application Ser. No. 2004/0029268, incorporated herein by reference, a medical device may be coated with a compound against which the antigen binding site of a multispecific antibody is directed, while another antigen binding site of the multispecific antibody is directed against a surface marker of endothelial progenitor cells. Preferably, the multispecific antibody is introduced into the bloodstream prior to angioplasty and implantation of the device. Recent advances have been made to devices whose therapeutic effect relies on the attachment and/or targeting of cells to a site of pathology, as noted in the above references. Targeting progenitor or stem cells to a site of pathology is of great interest in the scientific community because these cells can regenerate tissues that have deteriorated or sustained injury. Medical devices that target cells to a site of pathology are not limited to stents. Implants can be used to promote the binding of cells to repair damaged tissues. For example, implants have been used in tissue-engineered regeneration of damaged skeletal tissues. The implants provide binding sites for endogenous reparative cells (Caplan, Novartis Found Symp. 2003; 249:17-25). Implants have also been used to target adhesion of bone marrow cells (Torensma, et al., Clin Oral Implants Res. 2003;14 (5):569-577). Implants that release nerve growth factor, have been shown to target degenerated brain cells, which has generated considerable interest for the treatment of Alzheimer's Disease (Mahoney et al., Proc. Natl. Acad. Sci. 1999; 96: 4536-4539). Implantable collagen, used as an implantable medical device, has shown to increase the healing of skin wounds (Boyce, et al., Antimicrob Agents and Chemother. 1993; 37(9):1890-1895). Since the therapeutic effect of such devices is uncertain prior to clinical trial, an increasing need exists to measure how well they will promote cell adherence at a preclinical stage in development. Functionally testing the devices quantitatively in vitro, using living cells and physiologically relevant conditions, can predict effectiveness in vivo and provide useful feedback as to how the devices can be further developed and optimized. Such testing can foster technical advances in the design of such devices, leading to further improved patient outcome. Additionally, such methods are necessary to ensure quality control of manufacturing prior to use of such devices for implantation into humans.

### SUMMARY OF THE INVENTION

Accordingly, the present invention provides a method of quantifying the binding of cells to a medical device, the method comprising:
a) providing a medical device having at least one type of binding agent;
b) incubating the medical device with cells having at least one type of ligand expressing an affinity for the at least one type of binding agent;
c) labeling the cells that bind to the medical device;
d) detecting the cells bound to the medical device; and
e) determining the quantity of cells bound to the medical device.

If desired as a quality control procedure, the present invention further provides a method of determining the relative expression of the at least one ligand on the cell line having an affinity for the at least one binding agent.

The present invention also provides a method of quantifying the cell-binding properties of a medical device, the method comprising:
a) providing a medical device having at least one type of binding agent;
b) providing labeled cells having at least one type of ligand expressing an affinity for the at least one type of binding agent;
c) incubating the medical device with the labeled cells;
d) detecting the cells bound to the medical device; and
e) determining the quantity of cells that are bound to the medical device.

The method may include using a stent as the medical device. The stent is coated with an antibody and is incubated with a human cell line that constitutively expresses a ligand specific for the antibody. When the cells are bound to the stent, a fluorescent nucleic acid marker is used to label the cells. A fluorescence microplate reader measures the fluorescence of the cells bound to the medical device. The number of cells bound to the device is calculated by interpolation, using a standard curve of known numbers of fluorescently labeled cells.

Accordingly, a kit is provided for quantifying the cell-binding properties of a medical device, comprising at least one of the following: a binding agent; a cell line expressing affinity for the binding agent; a fluorescent marker; a binding agent that includes an antibody; a binding agent that includes a ligand; a cell line comprising a human cell line; a cell line comprising an animal cell line; a nucleic acid dye; blocking solutions, incubation and washing buffers; fixative solutions; cell culture media and supplements, instructions; and combinations thereof.

### BRIEF DESCRIPTION OF THE DRAWINGS

The invention is better understood by reference to the following description taken in combination with the accompanying drawings, of which:

FIG. 1 is a photograph of a prior art typical stent;

FIG. 2 is a graph showing a typical standard curve of fluorescence vs. cell numbers;

FIG. 3, top panel, is an illustration of the number of cells bound to stents of various binding capacities, as measured by fluorescence intensity of the assay, according to an embodiment of the present invention;

FIG. 3, bottom panel, shows photographs of the stents as indicated in FIG. 3, top panel.

### DETAILED DESCRIPTION OF THE INVENTION

The following description is meant to be illustrative only and not limiting. Other embodiments of this invention will be apparent to those of ordinary skill in the art in view of this description.

The present invention describes methods to quantify the binding of cells to a medical device. In particular, the invention relates to incubating, in a multi-well plate, a medical device, having a binding agent, with a single cell type that has an affinity for the binding agent on the medical device. Cells that are bound to the medical device are labeled with a marker. The marker is detected with a high-throughput fluorescence microplate reader, and the cells bound to the medical device are quantified.

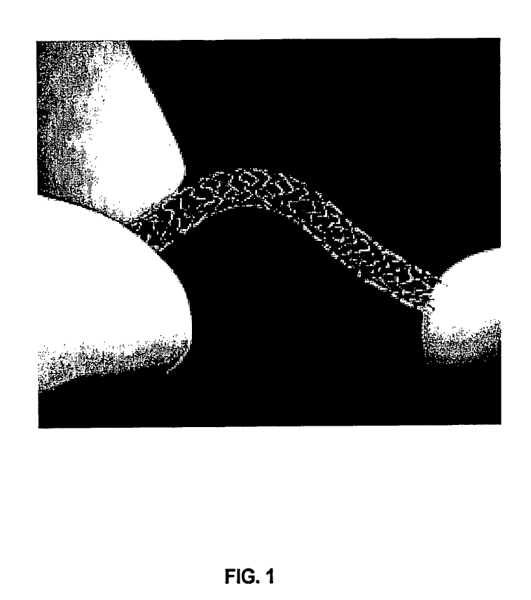

The present invention may be applied to a variety of medical devices that are used to repair or replace damaged tissues, organs, or other structures. Medical devices can include, but are not limited to, devices that come into contact with body tissues and fluids, for example, an implantable device. Medical devices of particular interest include stents, vascular grafts, synthetic grafts, or prostheses. Types of stents may include wire stents, tubular stents, mesh stents, or solid stents. FIG. 1 shows a typical stent. The term ‘medical devices’, as used herein, also includes devices used in medicine that may not come into contact with body tissues and fluids, for example, scaffolds and devices used in medical research. Substances applied on the medical device may include one or more binding agents. A binding agent refers to any substance that chemically or electrostatically bonds with another substance, preferably on a biological entity of interest. Among binding agents that may be used are antibodies, antigens, or combinations of the above. In a preferred embodiment, the medical device is a cardiovascular stent that has at least one binding agent, preferably an antibody, expressing affinity for a target antigen on the cell surface of precursor endothelial cells. The stent is typically incubated with blocking solution prior to incubation with the cells. The blocking step prevents the binding of cells by mechanisms other than antibody-antigen interaction (i.e., non-specific binding). The stent is then incubated with cells expressing the antigen of interest.

In the preferred embodiment herein described, these incubation conditions have been optimized for the following conditions: pre-blocking of devices, incubation vessel size, buffer or culture medium as incubating solution, different shaking protocols (flat vs. rotary vs. rolling, r.p.m., etc.), different temperatures of incubation, different times of incubation, amount/concentration of cells etc. Whereas conditions chosen in this particular case include blocking of nonspecific binding sites with 3% BSA for 15 minutes, incubation in 24-well plates for 1 hr at 37 degrees C. with rotary shaking using phosphate buffered saline (PBS) and 1% BSA as the diluent, and 500,000 cells per 400 microlitre volume during the incubation, it is recognized that these conditions may be further optimized as testing progresses with the current method, and indeed would need to be optimized anew for each new cell type, ligand type, binding agent type and device type on which the method may be used. Such optimizations are likely to have a large impact on cell binding to the device.

Once the cells are bound to the medical device, unbound cells are briefly rinsed off by dipping the device in a physiological buffer (i.e., PBS) and the bound cells may be fixed to the device with a fixative solution or by air drying, as well known to those skilled in the art, and labeled with a marker, preferably a fluorescent DNA-binding marker. Depending on the fixative solution used, the process to remove the fixative solution (i.e., air drying, rinsing, etc.), which is well known to those skilled in the art, may vary. The DNA-binding marker stains the cells with a fluorescent dye that binds to the DNA in the nuclei of the cells. In the preferred embodiment, fixation to and/or air drying stents may be necessary to avoid cell loss during steps preceding detection, however, in further embodiments of the assay using different devices, binding agents, or cell types, this step may prove unnecessary. Alternatively, labeled cells having at least one type of ligand expressing an affinity for the at least one type of binding agent may be provided that are incubated with the medical device.

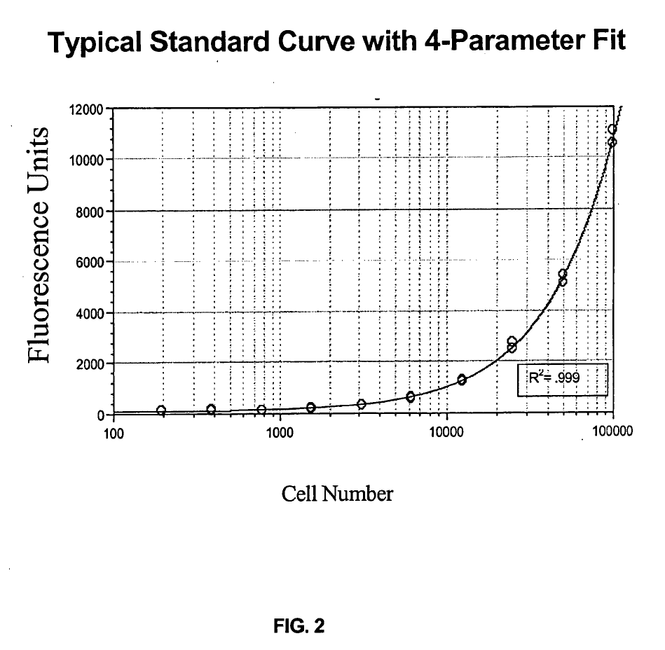

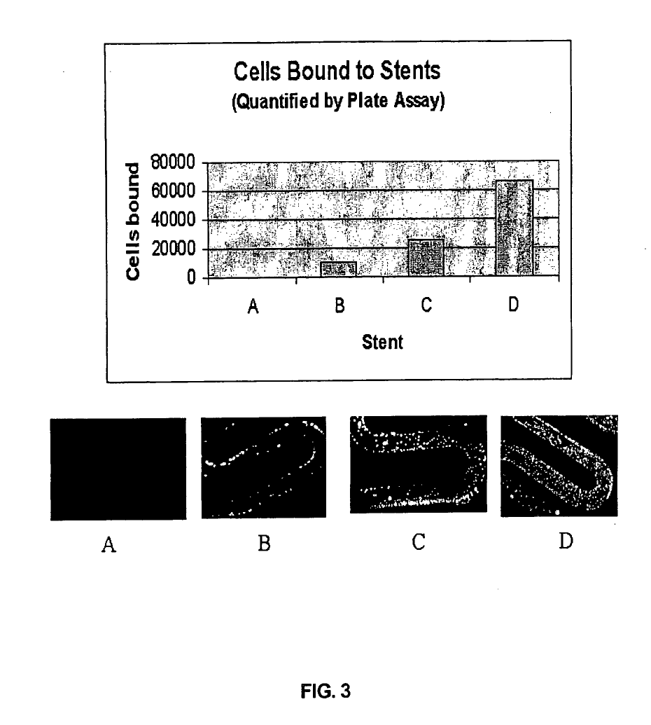

Many different DNA dyes are commercially available and an appropriate dye must be chosen to match the dye uptake characteristics of the particular cell type used in the assay, provide appropriate signal intensity for dynamic linear quantification according to the specifications of the particular assay, and avoid overlap with nonspecific spectral noise associated with other components of the particular assay. In the present embodiment, both DAPI and Hoechst dyes have been successfully used, but other nucleic acid dyes may also be used. Nucleic acid dyes may require permeabilization of the cells to enter; if so, the cells are permeabilized with a Triton-X-BSA solution according to permeabilization protocols known to those skilled in the art. Cellular dyes, other than nucleic acid dyes, may also be used as is known to those skilled in the art. The fluorescence of the assay is measured, in a high-throughput format, with a fluorescence microplate reader. In the present embodiment, a 96-well plate is used, but different sized plates may be used according to the size of the medical device and the capacities of the microplate reader. The microplate reader is set to excite and detect emissions of the fluorescently labeled nucleic acid with appropriate wavelength settings and cutoff filters. A standard of a known number of fluorescently labeled cells without a device is also added to the plate, so that the relationship between cell number and fluorescence units can be determined, and the quantity of cells bound to the medical device can be calculated by interpolation of a non-linear regression fit to the standard curve. FIG. 2 shows a typical standard curve. Fluorescence units are on the Y-axis, and cell numbers are on the X-axis. FIG. 3 illustrates that specificity and dose-response relationship in the microplate assay (according to the embodiment of the present invention) are well corroborated by microscopic evidence of cell binding; i.e., stents that bind no cells (A, bottom panel) show no signals in the assay (A, top panel), and stents that bind lower (B), intermediate (C), or higher (D) numbers of cells (bottom panel) produce correspondingly increased fluorescent signals in the microplate assay (top panel). Note that in the bottom panel of this figure, which shows fluorescence photographs, darkness indicates an absence of cells, whereas white points indicate the fluorescent nuclei of bound cells. Since cells may vary as to their expression of ligand specific to the binding agent due to cell culture conditions, quality control of the cell ligand expression may be performed on the batch of cells used in the assay. This assures that differences between assays in the number of cells bound to medical devices are related to the device and not to variability in cell surface expression. In the present embodiment, fluorescently labeled binding agent, the same agent that is on the medical device, is incubated without the device with a standard curve of known numbers of the same batch of cells that are used in the assay. The fluorescence is measured with a microplate reader, and the equation of the non-linear regression fit of the standard curve describes the relationship between cell number and fluorescence units. For a given lot of fluorescent binding agent used in various assays then, comparison of the mathematical attributes of the curve between assays suggests differences in cellular ligand expression for the binding agent between batches of cells used in the assays. Although cellular ligand expression may be verified in cells that have not had their DNA stained, in the present embodiment of the method, this quality control check of cell ligand expression is verified in the same standard curve of cells used to quantify cell number bound by devices, via implementation of a double staining protocol using two spectrally separated fluors (one DNA-binding dye, and the other attached to the cell surface antigen-binding agent) for a single standard curve. The applicant has developed such a double-staining method using an antibody labeled with phycoerythrin (PE-antibody) as a marker of membrane expression of the ligand used to bind cells to the medical device, and Hoechst 33342 to label the DNA of the cells. The method comprises blocking of nonspecific cell membrane antigens with an FBS (fetal bovine serum)—BSA solution, incubation with PE-antibody, washing of cells with PBS, incubation with Hoechst, further washing, counting of cells and seeding known numbers into replicate wells of a 96-well plate, such that an approximately 8-point standard curve is generated. Signals are then read from the plate as described above at two separate excitation/emission wavelength settings, the first one being the settings for detection of the nuclear marker and the second being those for detection of the surface antigen marker. Application of non-linear regression equations to the two readouts provide two standard curves: one for calculating number of cells bound to devices, and a second curve whose potential directional shifting, or changes in parameters such as maximum or 50% value serve as a quality control of the cell batch for expression of ligand. Should cellular ligand expression be verified in cells that have not had their DNA stained, a more restrictive set of quality control samples, such as low, medium, and/or high cell number samples may be analyzed. In this way, sample throughput capacity is maintained by maximizing availability of wells for sample rather than quality control analysis.

Controls that may be used in the assay to check for nonspecific cell binding to devices may include incubating the cell line with devices that do not possess a binding agent. Alternatively, controls may include devices with a binding agent in the presence of a different cell type that does not express affinity for the binding agent.

The assay developed is inventive in that it 1) directly measures binding interactions between cells and medical devices in a much higher throughput fashion than previous microscopic methods allow, 2) incorporates several nonobvious improvements over prior art assays as described in the following paragraphs, and 3) employs the actual medical device and not a surrogate piece of the device material (i.e., therefore, biologically relevant issues related to the structure of the device are incorporated into the method). Fluorescence plate reading, used as the endpoint measure of the present method, is certainly a widely used and established technology, however, the present method is novel with respect to standard plate assays for fluorescence reading in that the medical device itself is directly measured, contrasting with standard fluorescent plate assays, which measure biomarkers present in solution.

Furthermore, the geometry of the device as well as the light-blocking properties of stainless steel, combined with a measurement device (i.e., fluorescence microplate reader) where passage of different light wavelengths in and out of the sample matrix constitutes the signal, have provided technical challenges that have necessitated a number of optimizations of the assay that would not be part of a traditional fluorescence plate assay.

Plate assays commonly control for background fluorescence, sometimes referred to as autofluorescence, of solutions or the plate material itself by subtracting blanks from all other values. In this assay, background plate fluorescence cannot be subtracted from each well without creating error. Because the medical device may consist of a tube-like structure of coated stainless steel helical mesh, it interferes with light transmission to and from the plate surface in an uncontrollable and non-uniform way from well to well, and false negative signals of a widely variable range may result if plate-derived fluorescence is subtracted.

The applicant overcomes this problem with a nonobvious optimization by initially performing spectral scanning tests to identify the autofluorescence ranges of materials used in the assay, including but not limited to plates, solutions, cells, and medical devices. Then, a DNA marker with spectral characteristics that avoids the autofluorescence spectra of the assay material is chosen, and excitation and emission wavelengths and cutoff filters are set such that wavelength ranges for cell detection would not overlap with autofluorescence of empty or solution-filled wells. Therefore, no appreciable background (blank) fluorescence is detected in the assay, as illustrated in FIG. 3a, top panel.

Plate assays usually measure fluorescence that is dispersed throughout the solution present in the assay well, with the emission detector reading a single spot in the centre of each well. The principle relies on a uniform solution where fluorescence is the same in one spot as another, which is not the case for measurement of a medical device where fluorescence occurs only at spots of cell-binding to the device, and the device only occupies discrete areas of the well.

To inventively address this challenge, fluorescence signals are scanned from the entire well (9 points, evenly spaced throughout each well of the 96-well plate, and the maximum possible number of points with the applicant's instrumentation) rather than a single point. Also, white plates, which enhance reflection of signals, are used, rather than the black plates usually favored for fluorescence because of their lower backgrounds.

The present method uses a human cell line expressing the antigen of interest constitutively, rather than a mixed population of freshly isolated mononuclear cells which would contain only a small percentage of antigen-positive cells. By using a single cell type in the assay, as described in the present embodiment of the invention, the applicant inventively simplifies the fluorescent staining, relative to prior art fluorescent detection assays that were based on discriminating between different types of cell, immunocytochemically. In the present method, fluorescence serves as a label of all cells and therefore potential error in discrimination capacity is not a factor.

Compared to prior art assays using fresh blood cells, advantages of using a cell line include not needing to recruit blood donor subjects or do lengthy and variable preparations of human blood cells for each experiment, as well as avoidance of high inter-experimental variability that is due simply to inter-individual (i.e., donor) variability. Furthermore, cell lines are easily maintainable in culture and can be propagated indefinitely, providing a constant source of reproducible test material. Finally, the use of a cell line ensures that large numbers of antigen-positive cells are available to bind to the device, an improvement which allows output signals to be within the measurement range of a fluorescence microplate reader, and therefore permits the use of this high-throughput detection methodology, in which hundreds of samples can be read in minutes and accurately quantitated.

A significant improvement in quantitative capacity is brought to the present method with the incorporation of a standard curve. Results in previous assays were only meaningful when expressed relative to other sample groups analyzed in the same experiment, using the same batch of blood cells isolated from the same individual. Using the present method, the total cell numbers bound to devices can be calculated by nonlinear regression for each experiment, and different experiments done on different days by different operators using different batches of cells can be directly compared to each other.

While the embodiments of the invention disclosed are presently considered to be preferred, various changes and modifications can be made without departing from the scope of the invention. The disclosure is intended to be illustrative and not exhaustive. This description will suggest many variations and alternatives to one of ordinary skill in this art. All these alternatives and variations are intended to be included within the scope of the attached claims. Those familiar with the art may recognize other equivalents to the specific embodiments described which are also intended to be encompassed by the claims attached to the present embodiment.

1. A method of quantifying the cell-binding properties of a medical device, the method comprising:
a) providing a medical device having at least one type of binding agent;
b) incubating the medical device with cells having at least one type of ligand expressing an affinity for the at least one type of binding agent;
c) labeling the cells that bind to the medical device;
d) detecting the cells bound to the medical device; and
e) determining the quantity of cells that are bound to the medical device.

2. The method as claimed in claim 1 further comprising determining the relative expression of the at least one type of ligand on the cells having an affinity for the at least one type of binding agent.

3. The method as claimed in claim 1 or 2, wherein the method incorporates high throughput, screening.

4. The method as claimed in claim 1 or 2, wherein step (d) is effected by using a multi-well, high throughput microplate reader.

5. The method as claimed in claim 4 wherein the multi-well plate is a white, opaque 96-well plate.

6. The method as claimed in claim 1 or 2, wherein in step (c) labeling is effected by using at least one fluorescent marker.

7. The method as claimed in claim 6, wherein the at least one fluorescent marker is a nucleic acid dye.

8. The method as claimed in claim 6, wherein the spectral characteristics of the at least one fluorescent marker are well separated from the autofluorescence spectral ranges of materials used in the assay.

9. The method as claimed in claim 7, wherein the nucleic acid dye is DAPI.

10. The method as claimed in claim 7, wherein the nucleic acid dye is Hoechst 33342.

11. The method as claimed in claim 1 or 2, wherein in step (d) the detection is of at least one fluorescent marker of the cells bound to the medical device.

12. The method as claimed in claim 11, wherein the fluorescence of the fluorescent marker is measured at the excitation and emission wavelengths corresponding to the excitation and emission wavelengths of the fluorescent marker.

13. The method as claimed in claim 1 or 2, wherein the incubating in step (c) is effected in a multi-well plate.

14. The method as claimed in claim 13, wherein the multi-well plate is a 24-well plate.

15. The method as claimed in claim 1 or 2, which includes effecting step (e) of claim 1 by correlating the measured fluorescence with cell number by comparing to a multipoint control standard curve of known cell numbers, applying nonlinear regression analysis to the standard curve, then interpolating unknown values using the equation of the curve.

16. The method as claimed in claim 1 or 2, wherein after step (a) spectral scanning tests are performed to identify the autofluorescence ranges of materials used in the assay.

17. The method as claimed in claim 1 or 2, wherein after step (b) the cells are fixed with a fixing solution.

18. The method as claimed in claim 1 or 2, wherein after step (b) the cells are air dried.

19. The method as claimed in claim 1 or 2, wherein the medical device is a stent.

20. The method as claimed in claim 1 or 2, wherein the medical device is a scaffold.

21. The method as claimed in claim 1 or 2, wherein the medical device is a synthetic graft.

22. The method as claimed in claim 1 or 2, wherein the medical device is an implant.

23. The method as claimed in claim 1 or 2, wherein the at least one binding agent is a ligand.

24. The method as claimed in claim 1 or 2, wherein the at least one binding agent is an antibody.

25. The method as claimed in claim 1 or 2, wherein the cell line is human.

26. The method as claimed in claim 1 or 2, wherein the cell line is animal.

27. The method as claimed in claim 2, which includes effecting claim 2 by labeling the ligand, wherein labeling comprises:
a) providing the at least one binding agent that is fluorescently labeled;
b) providing a known number of the cells expressing a ligand for the at least one binding agent;
c) labeling the at least one cellular ligand of the known number of cells with the at least one fluorescently labeled binding agent;
d) detecting and quantifying the at least one fluorescently labeled binding agent bound to the cellular ligand, using a high-throughput fluorescence microplate reader;
e) determining the relative expression of the at least one cellular ligand on the current batch of cells by comparison of fluorescence intensity with that of previous batches of the same known numbers of cells.

28. The method as claimed in claim 27, wherein the at least one binding agent is a ligand.

29. The method as claimed in claim 27, wherein the at least one binding agent is an antibody.

30. The method as claimed in claim 27, wherein the fluorescent label of the at least one binding agent is phycoerythrin.

31. The method as claimed in claim 2, which includes effecting claim 2 by double labeling, wherein double labeling comprises:
a) providing the at least one binding agent that is fluorescently labeled;
b) providing a known number of the cells expressing a ligand forthe at least one binding agent;
c) labeling the at least one cellular ligand of the known number of cells with the at least one fluorescently labeled binding agent;
d) labeling the known number of cells with at least one fluorescent nudear marker,
e) detecting and quantifying the at least one fluorescent nuclear marker using a high-throughput fluorescence microplate reader, and
f) detecting and quantifying the at least one fluorescently labeled binding agent bound to the cellular ligand, using a high-throughput fluorescence microplate reader;
g) determining the relative expression of the at least one cellular ligand on the current batch of cells by comparison of fluorescence intensity with that of previous batches of the same known numbers of cells.

32. The method as claimed in claim 31, wherein the spectral characteristics of the fluorescent label of the at least one binding agent are well separated from the spectral characteristics of the at least one fluorescent nuclear marker.

33. The method as claimed in claim 31, wherein the at least one binding agent is a ligand.

34. The method as claimed in claim 31, wherein the at least one binding agent is an antibody.

35. The method as claimed in claim 31, wherein the fluorescent label of the at least one binding agent is phycoerythrin.

36. The method as claimed in claim 31, wherein the at least one fluorescent nuclear marker is Hoechst 33342.

37. A kit for quantifying the cell-binding properties of a medical device, comprising at least one of the following: a binding agent; a cell line expressing affinity for the binding agent; a fluorescent marker, a binding agent that includes an antibody; a binding agent that includes a ligand; a cell line comprising a human cell line; a cell line comprising an animal cell line; a nucleic acid dye; blocking solutions, incubation and washing buffers; fixative solutions; cell culture media and supplements; instructions; and combinations thereof.

38. The method of any of claims 1 to 36 wherein the cell is a from a cell line.

39. The method of any of claim 1 or 2 which includes effecting step (e) by determining the number of cells bound to the medical device at multiple points on the medical device.

40. A method of quantifying the cell-binding properties of a medical device, the method comprising:
a) providing a medical device having at least one type of binding agent;
b) providing labeled cells having at least one type of ligand expressing an affinity for the at least one type of binding agent;
c) incubating the medical device with the labeled cells;
d) detecting the cells bound to the medical device; and
e) determining the quantity of cells that are bound to the medical device.

41. The method as claimed in claim 40 further comprising determining the relative expression of the at least one type of ligand on the cells having an affinity for the at least one type of binding agent.
